(19) United States
(12) Patent Application Publication
(10) Pub. No.: US 2021/0318292 A1
(43) Pub. Date: Oct. 14, 2021

(54) System and Method to Detect Small Molecules

(71) Applicant: Sensorium Bio, Inc., San Francisco, CA (US)

(72) Inventors: Jean-Charles Neel, San Francisco, CA (US); Elias L. Rose, San Francisco, CA (US); Don Van Tran, Gilroy, CA (US)

(21) Appl. No.: 17/229,467

(22) Filed: Apr. 13, 2021

Related U.S. Application Data
(60) Provisional application No. 63/008,900, filed on Apr. 13, 2020.

(51) Int. Cl. G01N 33/50

(52) U.S. Cl. CPC G01N 33/5091; G01N 2458/30; G01N 2333/726

(57) ABSTRACT

The present invention relates generally to a system and a method to detect small molecules, also known as Volatile Organic Compounds (VOCs), in a fluid sample. The system and method can also be applied to diagnose a disease state of an organism by detecting a unique combination of metabolites produced by an organism.

### CROSS-REFERENCE TO RELATED APPLICATION

This application claims the benefit of U.S. Provisional Patent Application Ser. No. 63/008,900, entitled “System and Method to Detect Small Molecules,” filed on Apr. 13, 2020, the contents of which are incorporated by reference herein.

### BACKGROUND OF THE INVENTION

The present invention relates generally to a system and a method to detect small molecules, also known as Volatile Organic Compounds (VOCs), in a fluid sample. The system and method can also be applied to diagnose a disease state of an organism by detecting a unique combination of metabolites produced by an organism.

Cellular clusters act as capturing and sensing organs in living creatures. For example, the cluster of olfactory neurons in the nose feed into the olfactory bulb and then into the brain where the signals are processed to recognize individual and combinations of compounds detected in the nose. Likewise, retinal cells (cones and rods) in the eye detect light. Signals are fed directly into the brain for processing. Thus, live sensory cells can be used to detect molecules if the cells have the specific surface receptors that can interact with the molecule (olfactory cells) or stimulus (retinal cells) to produce a signal. In the case of a sensory neuronal cell such as the olfactory neurons, this signal is an electrical signal. In non-neuronal cells, the signal can be in the form of a chemical signal leading to the generation of an optically measurable signal.

Metabolites are the result of cellular processes and their relative levels are indicative of activities in the cells of the organism. Recognition of a combination of metabolites produced by the organism provides information about the status and the physiological changes of the organism. Thus, biomarkers derived from the metabolome are indicative of disease state (such as cancer) or medical state (such as diabetes). These can be used as diagnostics for a particular medical state (Zhang et al. 2012).

The present invention discloses the use of live non-neuron cells (also known as chemosensing cells) with specific surface receptors that interact with an intended target molecule in a fluid sample to generate an intracellular change which can be assessed through an optical readout, capturing the readout signal, and determining the presence or the absence of the target molecule in the sample. Using a combination of different cells with different receptors simultaneously allows the detection of a combination of target molecules, a metabolite fingerprint or profile, which can be unique to a specific disease state such as but is not limited to diabetes, cancer, and cardiac diseases.

These and other aspects and attributes of the present invention will be discussed with reference to the following drawings and accompanying specification.

### SUMMARY OF THE INVENTION

In an embodiment, the present invention discloses a system for detecting a target molecule in a fluid sample. The target molecule in the present invention is intended to be a water-soluble volatile organic molecule, also known as Volatile Organic Compounds (VOCs). As defined in the present invention, the VOCs are organic water-soluble molecules having molecular weight of about 1,000 g/mole or less. As used in the present invention, the target molecule is also referred to as a ligand, a biomarker, a marker or a metabolite, which can be used interchangeably.

The fluid sample comprises molecules dissolved or suspended in a fluid medium. The molecules in the sample may include macromolecules and/or small molecules as well as small water-soluble volatile organic molecules, which may or may not include the target molecule. The fluid sample can be a gaseous fluid, or a liquid fluid or a combination of gaseous and liquid fluid.

The fluid sample is introduced into the system through a receiver which serves as an intake of the fluid sample into the system. Once inside the system, a separator separates the water-soluble volatile organic molecules from the other molecules in the fluid medium as well as the fluid medium. The type of separator depends on whether the fluid sample is gaseous or liquid or a combination.

The system further comprises a plate with an optically clear bottom with a plurality of wells of live non-neuron cells, each well containing one or more live non-neuron cells covered by cell culture medium to keep the cells alive. Each cell has a plurality of a cell surface G-protein coupled receptor (GPCR), a G-protein and a reporter. The GPCR is capable of binding to the target molecule to trigger the G-protein to generate an intracellular secondary messenger signal which can be captured by the reporter to produce a change in emitted light (luminescence) or re-emitted light (fluorescence). Below the plate is a photosensor which detects the emitted light. The photosensor is connected to a computing device with a display for displaying the emitted light signal information. The increase of light signal indicates the presence of the target molecule and the absence of a light signal indicates the absence of the target molecule.

The GPCR, the G-protein or the reporter can be naturally existing in the non-neuron cell or one or more of them can be genetically engineered to be expressed by the cell. The GPCR that is naturally existing in the non-neuronal cell is known as an endogenous receptor. The GPCR that is genetically engineered to be expressed by the cell is known as an exogenous receptor. In a preferred embodiment, the GPCR receptor is an exogenous receptor. The cell may have both the endogenous GPCR and the exogenous GPCR. The GPCR. the G-protein or the reporter can be naturally occurring or synthetic.

In another embodiment, the present invention discloses a system for determining a disease state of a mammalian subject wherein the disease state exhibits a unique combination of water-soluble volatile organic marker metabolites (having molecule size of about 1,000 g/mole of less) present in a fluid sample from the subject, and the system comprising: (a) a receiver to receive the fluid sample into the system; (b) a separator separating the water-soluble volatile organic molecules from the fluid medium wherein the fluid medium is retained in the system or released from the system and the water-soluble volatile organic molecules are allowed to move forward within the system; (c) plate with an optically clear bottom with an array of wells each well having a single type of live non-neuron cells covered with a cell culture medium to keep the cells alive wherein: (i) the water-soluble volatile organic molecules are allowed to diffuse into the cell culture medium covering the cells; (ii) the live non-neuron cell having a surface GPCR capable of binding to the target molecule to produce a light emission in the presence of a G-protein and an appropriate reporter; (iii) the non-neuron cells separately express a GPCR responsive to metabolites exhibited by the disease state; and (iv) the cells in the array of wells represent all the GPCRs needed to detect the entire combination of the metabolites of the disease state; (c) a photosensor below the plate for detecting the light emission from the cells; and (d) a computing device connecting to the photosensor having a display for displaying the light emission information from each well wherein the presence of a light emission indicates the presence of the target molecule in the fluid sample and the presence of the entire combination of the marker metabolites indicate the presence of the disease state of the subject.

### BRIEF DESCRIPTION OF THE DRAWINGS

FIG. 1 is a schematic showing a side view of an embodiment of the system wherein the fluid sample is a gaseous sample. A vent is preferably in an open position when a button is pressed, and a subject then exhales air into a receiver of the system. The air travels from a proximal end of the tubing to a distal end of the tubing, passing through a resin located in a resin portion of the tubing wherein water-soluble volatile organic molecules are bound to the resin and excess air is released from the system. Preferably, a heating coil surrounds the resin portion of the tubing.

FIG. 2 is another side view of the embodiment of FIG. 1 wherein it is illustrated that the release of the button closes the vent.

FIG. 3 is another side view of the embodiment of FIG. 1 wherein the heating coil heats up the resin portion of the tubing to release water-soluble volatile organic molecules from the tubing and disperses into a cell culture medium with live non-neuron cells inside the wells in a well plate.

FIG. 4 a cross-sectional side view of an embodiment wherein the fluid sample is an exhaled breath sample comprising a gaseous phase containing small organic volatile molecules and a liquid phase of a bodily fluid such as mucus or saliva.

FIG. 5 is a sequence of images showing lung cancer metabolites detected in the present invention at various times.

FIGS. 6A and 6B are dose response curves of a luminescent assay. FIG. 6A is the detection of propylbenzene by OR1A1 and FIG. 6B is the detection of 1-hexanol by mOR256.

FIG. 7 is a dose response curve of various metabolites of a fluorescent assay.

### DETAILED DESCRIPTION OF THE INVENTION

While the invention is susceptible of embodiments in many different forms, there are shown in the drawings, and will be described herein in detail, specific embodiments thereof with the understanding that the present disclosure is to be considered as an exemplification of the principles of the invention and is not intended to limit the invention to the specific embodiments illustrated.

In an embodiment, the present invention discloses a system for detecting a target molecule in a fluid sample. The target molecule in the present invention is intended to be a water-soluble volatile organic molecule, also known as Volatile Organic Compound (VOC). As defined in the present invention, the VOCs are organic water-soluble molecules having a molecular weight of about 1,000 g/mole or less.

The fluid sample comprises molecules dissolved or suspended in a fluid medium. The molecules in the sample may include macromolecules and/or small molecules as well as small water-soluble VOCs, which may or may not include the target molecule. The fluid sample can be a gaseous fluid or a liquid fluid or a combination of gaseous fluid and liquid fluid. An example of the gaseous fluid sample is an exhaled breath condensate from a subject. Examples of liquid fluid samples include but are not limited to bodily fluids from a subject such as blood, saliva or urine, or an extract or a homogenate from an organic source such as a tissue or a plant or part of a plant.

An example of the combination of gaseous fluid and liquid fluid is an exhaled breath sample directly coming from a subject such as but is not limited to a human subject. The exhaled breath sample can be generated through the mouth, in which the sample may contain a combination of gaseous fluid from the breath as well as the liquid fluid of saliva. Alternatively, the breath may be generated through the nose, in which the sample may contain a combination of gaseous fluid from the breath as well as potential mucus from the nose or upper airways. Preferably, the breath sample is generated through exhalation of the breath through the mouth.

The fluid sample is introduced into the system through a receiver which serves as an intake of the fluid sample into the system. Once inside the system, a separator separates the water-soluble VOCs from the other molecules in the fluid medium as well as the fluid medium. The type of separator depends on whether the fluid sample is gaseous or liquid.

The system further comprises a plate with an optically clear bottom with a plurality of wells of live non-neuron cells, each well containing a plurality of live non-neuron cells covered by cell culture medium to keep the cells alive. Each cell has a plurality of cell surface chemosensing GPCR, a G-protein and a reporter. The GPCR is capable of binding to the target molecule resulting in the G-protein to trigger a cascade of intracellular events to generate an intracellular secondary messenger signal, which can be captured by the reporter to emit an optically measurable change.

In yet another preferred embodiment, the cells are cultured at ambient temperature under ambient air without requiring incubation of the cells at 37° C. and 5% CO₂ by using a culture medium buffered by high buffering capacity carbon dioxide-independent buffer to maintain a pH of from about 7 to about 8 in the medium throughout the culture.

Culturing the cells at ambient temperature without incubation at 37° C. and 5% CO₂ has been disclosed in detail in US Provisional Patent Application entitled “Cell Culture System” Ser. No. 63/157,445, filed on Mar. 5, 2021 which is incorporated by reference herein in its entirety. The cell culture system disclosed is tightly sealed with a gas impermeable film to prevent fluid loss during the culture. After removing the gas impermeable film, the cell culture described is ready to be used in the present invention to detect the target molecule.

One or all of the GPCR, the G-protein or the reporter can be naturally existing with the non-neuron cell or they can be genetically engineered to be expressed by the cell. The GPCR that is naturally existing in the non-neuronal cell is known as an endogenous receptor. The GPCR that is genetically engineered to be expressed by the cell is known as an exogenous receptor. In a preferred embodiment, the GPCR receptor is an exogenous receptor. The cell may have both the endogenous GPCR and the exogenous GPCR. While many types of cells have natural GPCRs and G-proteins, most of the cells do not have the reporter, which has to be genetically engineered to be expressed by the cell if the cell does not naturally have the reporter. In the present invention, the GPCR is referred to as a cell surface receptor. Another term for the cell surface receptor is transmembrane receptor, which are used interchangeably in the present invention.

As used herewith, “G-protein-coupled receptor (GPCRs)”, also known as “seven-transmembrane domain receptors”, “7TM receptors”, “heptahelical receptors”, “serpentine receptors”, and “G protein-linked receptors (GPLR)”, designate a large protein family of receptors that sense molecules outside the cell and activate, inside the cell, signal transductions pathways and, ultimately, cellular responses. GPCRs are found in eukaryotes, including yeast and animals. The ligands that bind and activate these receptors are typically small in size and they include light sensitive compounds, odors, pheromones, hormones, cytokines and neurotransmitters, and vary in size from small molecules to peptides to large proteins. The olfactory neurons specifically express GPCRs to detect odors and taste neurons specifically express GPCRs to detect taste. Gustatory sensory neurons do not send axonal projections directly to the brain. Both types of olfactory and gustatory GPCRs are acceptable in the present invention.

The GPCR used in the present invention can be any GPCR from any species or cell types, or it can be modified from a natural GPCR to improve its functionality, such as but are not limited to increasing sensitivity and selectivity. GPCR modification can lead to changes in its binding to the target molecule or modulating selectivity for a targeted ligand. A preferred GPCR is an olfactory GPCR from humans or rodents.

It is critical that the non-neuron cells used in the present invention are able to express functional GPCRs. An example of the non-neuron cell used in the present invention is the Hana3A cell, which is a human embryonic kidney-derived cell that has been modified to express functional olfactory receptors. Hana3A cells have been engineered to have the internal signaling pathways needed for transmission of the binding signal from the odorant receptors. The modification includes but is not limited to the addition of Receptor Transport Proteins (RTP1, RTP2), Receptor Expression Enhancer Protein (REEP1, REEP2), Ric8b and GalphaOlf genes naturally found in olfactory neurons. Shorter versions of RTP proteins can also be used to confer the same property. Other non-neuronal cells can also be so engineered to have the same internal signaling pathways as the Hana3A cells using a similar engineering process. Without the modifications, it has not been shown to be able to successfully express functional olfactory receptors in a heterologous model (non-olfactory neuron). Detailed description of how the Hana3A cells are made has been disclosed by Saito H. et al. (Cell. 2004 Nov. 24; 119(5):679-91) which is herein incorporated by reference in its entirety. Hana3A cells have also been disclosed in U.S. Pat. Nos. 7,879,565, 7,838,288 and 7,691,592. Hana3A Accession No. CVCL_RW32.

In one embodiment, the non-neuron cells of the invention are non-neuronal somatic cells.

In a specific embodiment, the cells are mammalian cells. In a more specific embodiment, the non-neuron cells are human cells. The non-neuron cells can be cells that have been cultured in vitro, or cells that are freshly isolated from an animal. In one embodiment, the cells are epithelial cells, fibroblasts, melanocytes, keratinocytes, adipocytes, or Langerhans cells. Methods for preparing various non-neuron cell types are known in the art. The non-neuron cells can be obtained from a person or animal by invasive or non-invasive means. In one embodiment, cells are obtained by way of a biopsy. The non-neuron cells can be from a patient having a disease or condition.

As used herewith, the terms “olfactory receptors” designate the receptors expressed in the cell membranes of olfactory sensory neurons responsible for the detection of chemical cues. Activated olfactory receptors are the initial player in a signal transduction cascade which ultimately produces a nerve impulse which is transmitted to the brain. Most of these receptors are members of the GPCR superfamily. The olfactory receptors form a multigene family consisting of about 400 potentially functional genes in humans and about 1250 genes in mice. Olfactory receptors are generally categorized, in mammals, into several receptor families including odorant receptors (ORs), vomeronasal receptors (V1Rs and V2Rs), trace amine-associated receptors (TAARs), formyl peptide receptors (FPRs), and the membrane guanylyl cyclase GC-D.

In specific embodiments, the one or more odorant receptor is selected from the group consisting of MOR129, MOR103, olfr476, olfr491, olfr1104, olfr502, olfr1062, olfr919, olfr079, olfr876, olfr556, olfr979, olfr962, olfr145, olfr889, olfr1484, olfr978, olfr1512, olfr1411, olfr109, olfr1377, olfr124, olfr992, olfr549, olfr1364, olfr1370, olfr90, olfr1093, olfr167, olfr211, olfr2(17), OR-S6, Olfr62, S6/79, S18, S46, S50, MOR23-1, MOR31-4, MOR31-6, MOR32-5 and MOR32-11.

In other specific embodiments, the one or more odorant receptor is selected from the group consisting of OR10A2, OR13C8, OR2AG2, OR2T8, OR4M2, OR52L1, OR5M3, OR7G2, OR10A3, OR13C9, OR2AJ1, OR2V1, OR4N2, OR52M1, OR5M8, OR7G3, OR10A4, OR13D1, OR2AK2, OR2V2, OR4N4, OR52N1, OR5M9, OR8A1, OR10A5, OR13F1, OR2AP1, OR2W1, OR4N5, OR52N2, OR5P2, OR8B12, OR10A6, OR13G1, OR2AT4, OR2W3, OR4P4, OR52N4, OR5P3, OR8B2, OR10A7, OR13H1, OR2B11, OR2Y1, OR4Q3, OR52N5, OR5R1, OR8B3, OR10AD1, OR13J1, OR2B2, OR2Z1, OR4S1, OR52R1, OR5T1, OR8B4, OR10AG1, OR14A16, OR283, OR3A1, OR4S2, OR52W1, OR5T2, OR8B8, OR10C1, OR14A2, OR2B6, OR3A2, OR4X1, OR56A1, OR5T3, OR8D1, OR103, OR14C36, OR2C1, OR3A3, OR4X2, OR56A3, OR5V1, OR8D2, OR10G2, OR141I1, OR2C3, OR4A1S, OR51A2, OR56A4, OR5W2, OR8D4, OR10G3, OR14J1, OR2D2, OR4A16, OR51A4, OR56B1, OR6A2, OR8G1, OR10G4, OR14K1, OR2D3, OR4A47, OR51A7, OR56B3P, OR6B1, OR8G5, OR10G6, OR1A1, OR2F1, OR4A5, OR51B2, OR56B4, OR682, OR8H1, OR10G7, OR1A2, OR2F2. OR4B1, OR51B4, OR5A1, OR6B3, OR8H2, OR10G8, OR1B1, OR2G2, OR4C11, OR51B5, OR5A2, OR6C1, OR8H3, OR10G9, OR1C1, OR2G3, OR4C12, OR51B6, OR5AC2, OR6C2, OR812, OR1OH1, OR1D2, OR2G6, OR4C13, OR51D1, OR5AK2, OR6C3, OR8J1, OR10H2, OR1D5, OR2H1, OR4C15, OR51E1, OR5AN1, OR6C4, OR8J3, OR10H3, OR1E1, OR2H2, OR4C16, OR51E2, OR5AP2, OR6C6, OR8K1, OR10H4, OR1E2, OR2J1, OR4C3, OR51F1, OR5AR1, OR6C65, OR8K3, OR1OH5, OR1F1, OR2J2, OR4C46, OR51F2, OR5AS1, OR6C68, OR8K5, OR10J1, OR1G1, OR2J3, OR4C5, OR51G1, OR5AU1, OR6C70, OR8S1, OR10J3, OR1I, OR2K2, OR4C6, OR51G2, OR5B12, OR6C74, OR8U1, OR10J5, OR1J, OR2L13, OR4D1, OR51H1P, OR5B17, OR6C75, OR8U9, OR10K1, OR1J2, OR2L2, OR4D10, OR51I1, OR5B2, OR6C76, OR9A2, OR10K2, OR1J4, OR2L3, OR4D11, OR5112, OR5B21, OR6F1, OR9A4, OR10P, OR1K1, OR2L5, OR4D2, OR51L1, OR5B3, OR6J, OR9G1, OR10Q1, OR1L1, OR2L8, OR4D5, OR51M1, OR5C1, OR6K2, OR9G4, OR10R2, OR1L3, OR2M2, OR4D6, OR51Q1, OR5D13, OR6K3, OR9G9, OR10S1, OR1L4, OR2M3, OR4D9, OR51S1, OR5D14, OR6K6, OR9I1, OR10T2, OR1L6, OR2M4, OR4E2, OR51T1, OR5D16, OR6M1, OR9K2, OR10V1, OR1L8, OR2M5, OR4F15, OR51V1, OR5D18, OR6N1, OR9Q1, OR10W1, OR1M1, OR2M7, OR4F16, OR52A1, OR5F1, OR6N2, OR9Q2, OR10X1, OR1N1, OR2S2, OR4F17, OR52A5, OR5H1, OR6P1, OR10Z1, OR1N2, OR2T1, OR4F21, OR52B1P, OR5H14, OR6Q1, OR11A1, OR1Q1, OR2T10, OR4F29, OR52B2, OR5H15, OR6S1, OR11G2, OR1S1, OR2T11, OR4F3, OR52B4, OR5H2, OR6T1, OR11H1, OR1S2, OR2T12, OR4F4, OR52B6, OR5H6, OR6V1, OR11H12, OR2A1, OR2T2, OR4F5, OR52D1, OR5I1, OR6X1, OR11H4, OR2A12, OR2T27, OR4F6, OR52E2, OR5J2, OR6Y1, OR11H6, OR2A14, OR2T29, OR4K1, OR52E4, OR5K1, OR7A10, OR11L1, OR2A2, OR2T3, OR4K13, OR52E6, OR5K2, OR7A17, OR12D2, OR2A25, OR2T33, OR4K14, OR52E8, OR5K3, OR7A5, OR12D3, OR2A4, OR2T34, OR4K15, OR52H1, OR5K4, OR7C1, OR13A1, OR2A42, OR2T35, OR4K17, OR5211, OR5L1, OR7C2, OR13C2, OR2A5, OR2T4, OR4K2, OR52I2, OR5L2, OR7D2, OR13C3, OR2A7, OR2T5, OR4K5, OR52J3, OR5M1, OR7D4, OR13C4, OR2AE1, OR2T6, OR4L1, OR52K1, OR5M10, OR7E24, OR13C5, OR2AG1, OR2T7, OR4M1, OR52K2, OR5M11, OR7G1; and/or any variant thereof.

At present, there is limited information regarding which GPCR binds to which ligand or target molecule. However, the GPCRs can be screened to determine which specific target molecules bind to which GPCR. The screening process is well-known in the an and has been described in detail by H. Saito el al. (Sci Signal, 2009 Mar. 3: 2(60):ra9. doi: 10:1126/scisignal.20000016).

G-proteins in the present invention that associate with GPCRs are specialized proteins with the ability to bind the nucleotides guanosine triphosphate (GTP) and guanosine diphosphate (GDP). They are heterotrimeric, meaning they have three different subunits: an alpha (α) subunit, a beta (β) subunit, and a gamma (γ) subunit.

Upon ligand binding, the transmembrane G-protein coupled receptor undergoes a conformational change which leads to the activation of the G-protein component. The G-protein in turn activates other cellular components depending on the type of G-protein activated by the receptor. As the ligand (VOC metabolite in our case) binds to the extracellular part of the GPCR, this event triggers a conformational change in the activated receptor which causes the receptor to activate the coupled G-protein. The G-protein is a protein trimer composed of alpha-, beta- and gamma subunits. Upon activation by the receptor, the G-protein subunits separate, and the alpha subunit separate from the beta-gamma dimer. The alpha subunit initially part of the membrane bound G-protein trimer is then free to move into the cytosol to act as a receptor effector. The activated G-protein subunits each mediate part of the G-protein effects. Depending on the nature of the coupled G-protein, the receptor activation leads to a range of downstream effects including increases in cytosolic cAMP and Ca²⁺ levels. These changes can be monitored using an optical reporter system.

Olfactory receptors activate olfactory-specific G_{olf}-proteins which are stimulatory G₅α-like proteins. The receptor-mediated Gif-protein activation leads to a G_{olf}-mediated activation of another cellular component Adenylate Cyclase which proceeds to convert cellular adenosine triphosphate (ATP) into cyclic adenosine monophosphate (cAMP) messenger molecules which start accumulating up in the cytosol. Once a sufficient cAMP concentration threshold is reached, specific cAMP-responsive Ca²⁺ channels open in the cell outer membrane allowing extracellular Ca²⁺ to enter. At this point, extracellular Ca²⁺ flows into the cell mixing with intracellular Ca²⁺ mobilization leading to concentration increase.

The above cascade of events is entirely initiated by the binding of the ligand to the receptor and as such, using an intracellular reporter of the receptor activity allows one to indirectly infer information regarding the presence or absence of the ligand in the extracellular environment the cells are in immediate contact with.

Tracking the cellular concentration of cAMP can be performed using a commercial genetically-encoded cAMP reporter. Upon binding to cAMP, the cAMP reporter undergoes a change of conformation which leads to the increase of an enzymatic luciferase activity which can lead to the production of light if a specific luciferin chemical is provided at that point.

An example of the cAMP reporter is the Dual Luciferase Reporter assay system which is commercialized by Promega Corporation (Madison, Wis.) and described in detail by Binkowski, B. et al. (ACS Chem Biol. 011 Nov. 18; 6(11):1193-7.doi: 10.1021/cb200248h. Epub 2011 Sep. 22). In brief, as metabolites induce the activation of G-protein coupled receptors, these receptors in turn activate the G-protein they are preferentially coupled with. In the case of olfactory GPCRs, the G-protein is a stimulatory type-S G-protein which activates a cell membrane bound enzyme called Adenylyl Cyclase type-3 (AC3). AC3 starts converting cytosolic ATP into cAMP, which acts as a secondary messenger. Indeed, cAMP is typically entirely absent from the cytosol and its presence is a strong indicator of GPCR activation. The accumulated cAMP triggers the translocation to the nucleus of a cytosolic protein called cAMP-response element binding protein (CREBP). As a response to cAMP, this protein moves to the nucleus where it binds onto the DNA and induces activation of specific genes. Using a synthetic genetic construct which contains an CREBP inducible Firefly luciferase, a luminescent readout upon GPCR activation by a metabolite is possible.

Tracking the cellular concentration of Ca²⁺ can be performed using a highly selective commercial Ca²⁺-responsive fluorescent dye such as Fluo-4 (Biotium Inc., Hayward, Calif.) or Fura-2 (Sigma, St. Louis, Mo.). Upon Ca²⁺ binding, these dyes undergo a change in fluorescence which can be measured optically.

Tracking the cellular concentration of Ca²⁺ can be performed using a genetically-encoded Ca²⁺ reporter such as GCaMP consisting of a fusion between a split Green Fluorescent Protein (GFP), calmodulin (a Ca²⁺-modulated protein) and M13, a peptide sequence from myosin light chain kinase. Upon binding of Ca²⁺ to the calmodulin component of GCaMP, the Ca²⁺ reporter undergoes a change of confirmation which leads to the increase of a fluorescence of GFP. The detail of using GCaMPs as calcium reporters has been described in detail by Dana, H. et al. (Nature Methods: 16, 649-657(2019)).

Below the optically clear bottom of the plate is an objective which focuses and directs the emitted light to a photosensor which detects the emitted light. An example of the objective is the 5× EO HR Infinity Corrected Objective (Barrington, N.J.). The photosensor captures the focused light and converts to digital signal. The photosensor is also known as an optical sensor or an optical photosensor, which can be used interchangeably. The photosensor is connected to a computing device with a display for displaying the digital signal. The presence of a light signal indicates the presence of the target molecule and the absence of a light signal indicates the absence of the target molecule. An example of the photosensor is the Basler Ace acA4112-20uc USB 3.0 Color Camera (Barrington, N.J.). In the embodiments wherein the light signal is fluorescence, the system further comprises an excitation light source which produces excitation light to trigger the biological fluorescence of the Ca²⁺ reporter. An example of the excitation light source is 470 nm, High Intensity Coaxial Spotlight (Barrington, N.J.). A filter such as 520 nm CWL, 25 mm Dia., Hard Coated OD 4.0 10 nm BandPass filter (Barrington, N.J.) can be used with the excitation light source to block out the excitation light to allow only the desired excitation light through.

In a preferred embodiment, the emitted light signal is a fluorescence light with the calcium reporter. In this embodiment, a continuous or pulsed light source (typical wavelength of about 395 to about 480 nm) illuminates the cells briefly for about 10 to 500 msec and an increase in fluorescent light is re-emitted (around 509 nm). For the genetically-encoded calcium reporter is a GCaMP7s reporter, the excitation peak wavelength is about 450 nm and emission peak wavelength is about 515 nm. This indicates cellular change as a result of the binding of the target molecule to the receptor. The GCaMP7 is a family of calcium reporters. For example, the genetic construct GCaMP7s is a Ca²⁺ sensor that confers an increase in fluorescence following which can be measured with an appropriate filter to block off incoming light and track re-emitted light. The continuous or pulsed re-emitted light with a peak at 509 nm is not typically directional and after going through a filter to remove non-specific wavelengths is collected through a gradient-indexed lens to collimate onto a sensor such as a Sony IMX2644 CMOS in a Basler acA2440-20 gm camera. The photosensor collects the re-emitted light at a rate of about 20-30 frames per second depending on pixel binning.

In yet another embodiment, the emitted light is luminescent light when luciferase is used as a cAMP reporter. This embodiment does not require an excitation light source. An interaction of the target molecule with the receptor triggers a chemo-detection event which further triggers the activation of an endogenous enzymatic luciferase activity which can be interrogated by supplementing the media with a cell-permeable enzymatic substrate that can emit light upon degradation. Alternatively, the substrate can be generated endogenously and processed by the enzymatic activity. For reading the luminescent signal, a photosensor alone is sufficient to track the amount of light produced. Examples of relevant wavelengths for luminescent cellular responses could be around 480 nm (Renilla luciferase), around 565 nm (Firefly luciferase) and around 460 nm (NanoLuc luciferase).

The amount of light captured by the photosensor in both the fluorescent readout and the luminescent readout is proportional to the amount of the target molecule as demonstrated in Examples 2 and 3 below.

The initial steps in the intracellular cascade after the binding of the target molecule to the receptor in the present invention is an accumulation of cAMP and Ca²⁺. The rate of cAMP and Ca²⁺ accumulation in the cytosol is influenced by the affinity of the receptor for the target molecule binding to it. As the output signal is affected by cAMP or Ca²⁺ accumulation, it follows that two different cell populations each expressing a chemoreceptor with a different affinity for the molecule of interest would have an output response with a different time constant. By tracking not only the patterns of chemoreceptor-expressing cell populations but also the time patterns of this response, it may be possible to further characterize the sample composition.

In an embodiment wherein the fluid sample is a gaseous fluid sample, the separator comprises: (a) a tubing having a proximal end and a distal end and wherein the proximal end serves as the receiver or is connected to the receiver and the distal end is situated above the optically clear bottom of the plate with a plurality of wells of cells; (b) a resin portion of the tubing situated at the distal end of the tubing filled with a resin within the tubing and the resin having the capability of trapping water-soluble volatile organic molecules; (c) a vent below the resin portion of the tubing and above the plate and the vent having an open position and a close position wherein when the vent is in the close position, the gaseous sample is kept within the tubing and passes through resin wherein the water-soluble volatile organic molecules are bound to the resin and when the vent is in the open position the gaseous fluid is released from the system; and (d) heating coil surrounding the resin portion of the tubing wherein the water-soluble volatile organic molecules are released from the resin to disperse into the cell culture medium in the wells when the heating coil is turned on and when the vent is in the close position.

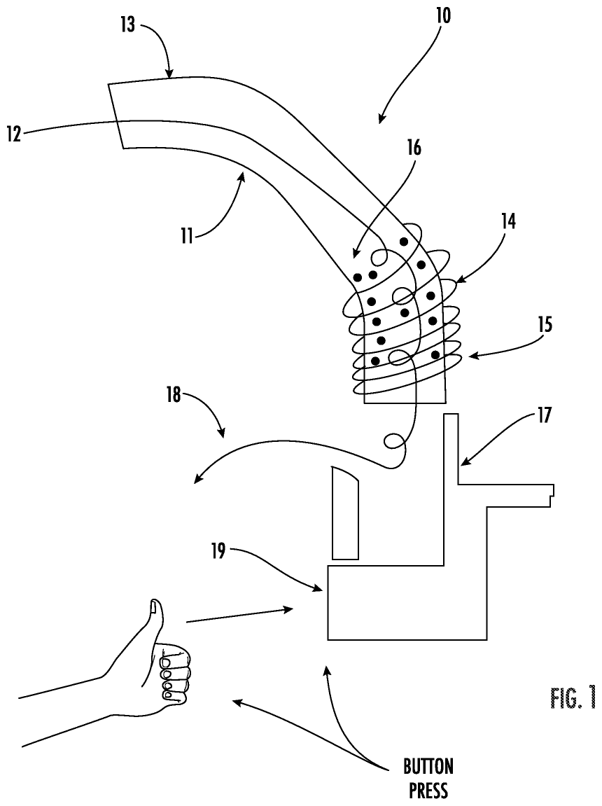

FIGS. 1, 2 and 3 are schematic drawings illustrating the separator of this embodiment. The drawings are for illustrative purposes and are not drawn to proportion or scale. FIG. 1 shows the separator 10. The subject exhales through the mouth or the nose into the proximal end 13 of the tubing 11 and simultaneously presses the button 19. The exhaled breath sample contains both the small volatile organic molecules as well as other larger molecules such as macromolecules. When the button 19 is pressed, vent 17 is in a closed position to allow exhaled breath 12 to enter into the tubing 11 of the separator 10. The breath sample travels along the tubing 11 towards the distal end of the tubing 15 and passes through resin 16 located at the distal end of the tubing 15. The small volatile organic molecules are adsorbed by the resin 16 while the other molecules are released from the system through the vent 17 in the closed position.

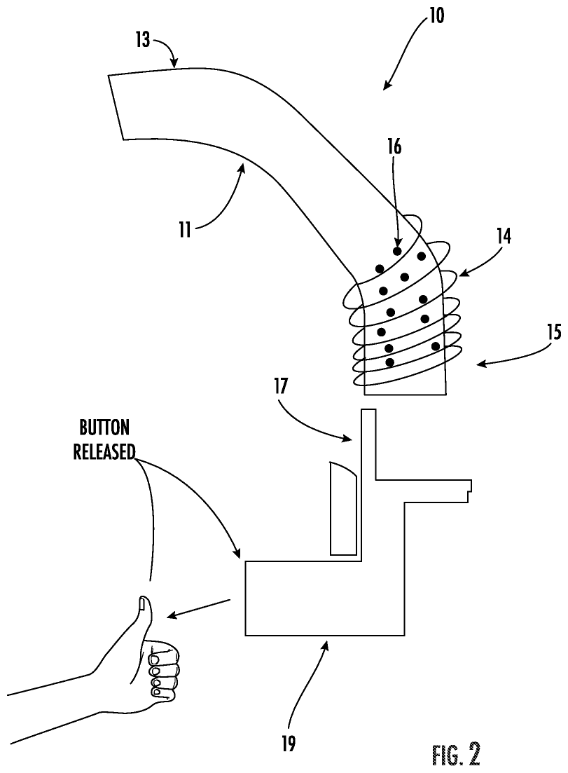

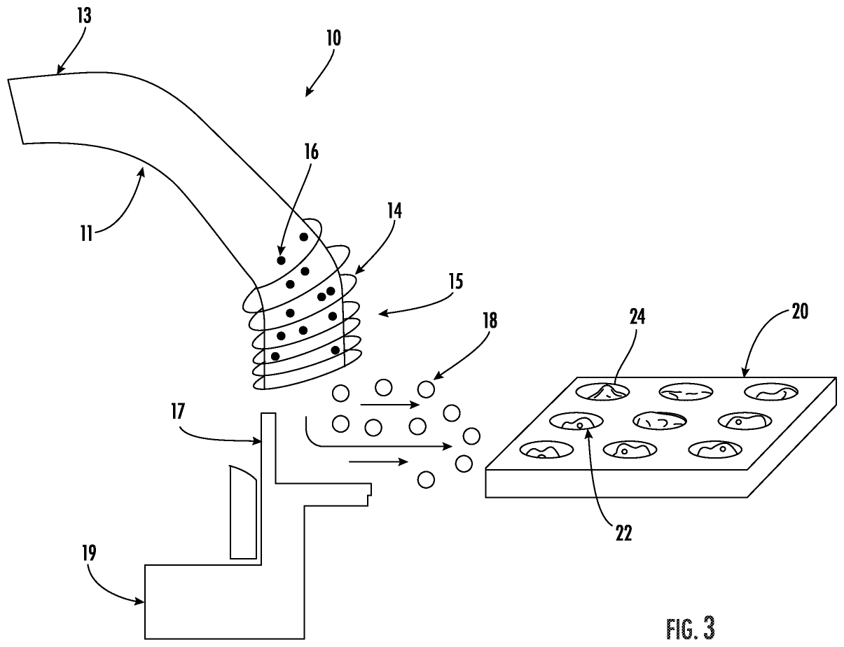

FIGS. 2 and 3 are the same embodiment as FIG. 1 wherein the subject releases the button 19 causing the vent 17 to be in an open position. The distal end of the tubing 15 where the resin 16 is located is surrounded by a heating coil 14 on the outside of the tubing 11. The heating coil 14 is connected to a power source (not shown). When the heating coil 14 is turned on, the temperature of the heating coil 14 heats up the resin 16 to release the small volatile organic molecules 18 onto the cells 24 in the wells 22 in the plate 20.

An example of the resin for the present invention to trap the small molecules is a TENAX® resin. To release the small volatile molecules from the resin, the heating coil heats up the resin to a temperature higher than the evaporation temperature of the adsorbed small molecules of interest to allow it to desorb from the resin and return to the gas phase. Once the resin has reached the required temperature, the duration of the heating is likely not to exceed 2 seconds.

In yet another embodiment, the fluid sample is a liquid, and the separator is a porous barrier which allows the water-soluble volatile organic molecules to pass through the porous membrane to reach the cell culture medium and subsequently interacting with the surface cell receptors of the live non-neuron cells while the liquid medium is excluded from reaching the cells. Examples of porous barriers include but not limited to a porous PTFE membrane or a PEG-based hydrogel allowing for compounds to interface with the live non-neural cells while retaining liquid media.

In yet a further embodiment, the fluid sample is a gaseous sample from an exhaled breath of a mammalian subject wherein the sample comprises a gaseous phase containing small volatile organic molecules (and possibly other larger molecules) and a liquid phase of bodily fluid. The exhaled breath can be generated through the nose or the mouth of the subject. It is anticipated that multiple cycles of inhalation and exhalation are needed in order to provide an adequate quantity of samples for the assay. In the case of the exhaled breath being through the nose, the bodily fluid is mucus. In the case of the exhaled breath being through the mouth, the bodily fluid is saliva. The fluid phase needs to be separated from the gaseous phase of the breath sample.

The separator in this embodiment comprises: (a) a tubular structure having a proximal end and a distal end and the tubular structure provides a convoluted path for the passage of the breath sample, the tubular structure comprises: (i) a receiving section at the proximal end of the tubular structure to receive the breath sample from the subject or is connected to a receiver of the system; (ii) an inhaled air inlet within the receiving section to let air into the tubular structure to provide air for the subject when the subject is inhaling thereby creating negative pressure; (iii) a first downward section allowing the breath sample to move down; (iv) a trapping section to trap the liquid phase from the breath sample which has moved down from the first downward section; (v) a vent between the first downward section and the trapping section, the vent being in a close position when the individual is inhaling and in an open position when the individual is exhaling to allow the exhaled breath sample to move along the first downward section and through the open vent into the trapping section; (vi) an upward section allowing the gaseous phase of the breath sample to move upward leaving the liquid phase of the breath sample behind within the trapping section; and (vii) a second downward section at the distal end of the tubular structure allowing the gaseous phase of the breath sample to move down; (b) a condensation chamber connecting to the second downward section of the tubular structure; (c) an exhaled air outlet in the condensation chamber to allow excess gaseous phase of the exhaled breath sample to escape from the tubular structure and providing a pull to pull the exhaled breath sample along the tubular structure; (d) a microcondenser in the condensation chamber to condense the gaseous phase of the breath sample into liquid droplets; (d) a cooling system below the microcondenser to lower the temperature of the microcondenser to facilitate the condensation of the gaseous phase of the breath sample on the microcondenser; and (e) a condensation droplet collecting tubing to collect the liquid droplets from the microcondenser, move the droplets along the collecting tubing and release the droplets from the tubing wherein the moving of the droplets in the droplet collecting tubing and releasing the droplets from the droplet collecting tubing is by means of an active push.

The microcondenser in this embodiment is well known in the art and has been disclosed by Davis, C. et al. in U.S. Pat. Nos. 9,398,881 B2, 10,067,119 B2 and 10,111,606 B2, the relevant portions of which are incorporated by reference.

The cooling effect of the cooling system in this embodiment can be provided by, but is not limited to, ice, dry ice, a fan or a Peltier cooler. In a preferred embodiment, the cooling system is a Peltier cooler, which is also known as a Peltier device, Peltier heat pump, solid state refrigerator, thermoelectric cooler or thermoelectric battery. It is an electronic solid-state active heat pump which transfers heat from the microcondenser.

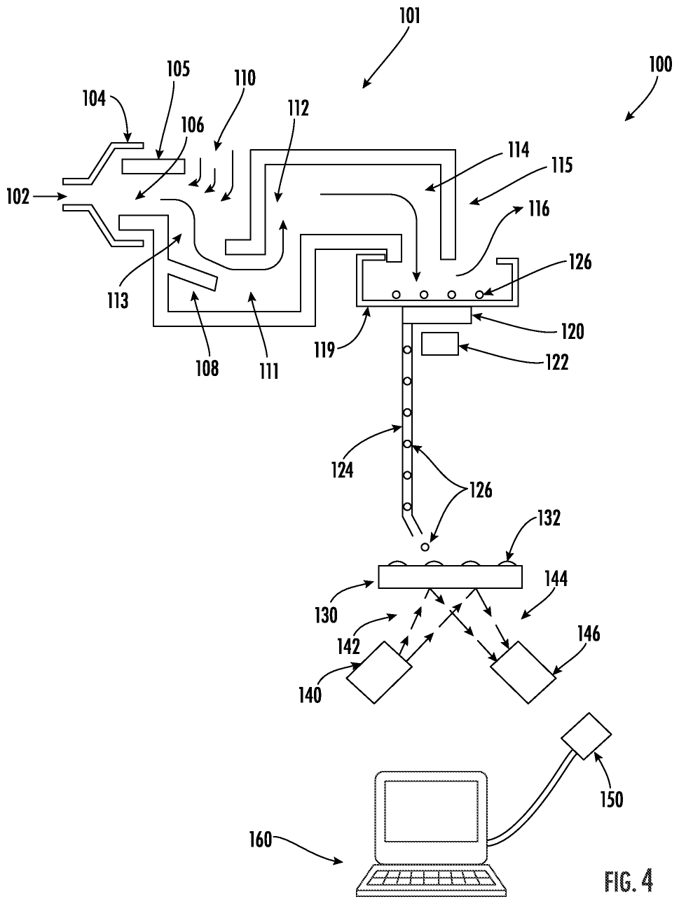

This embodiment is exemplified and illustrated in FIG. 4 which is a schematic cross section of the system 100. The drawing is for illustration purposes and is not drawn in proportion or scale. The separator 101 of the system 100 is a tubular structure to provide a convoluted path for the exhaled breath sample, the tubular structure 101 having a proximal end 105 and a distal end 115. A receiving section 106 is situated at the proximal end 105, which serves as a receiver to receive the exhaled breath 102 from a subject. Alternatively, the receiving section 106 is connected to a receiver 104. Within the receiving section 106 is an inhaled air inlet 110 to let air into the receiving section 102 to provide air to the subject when the subject (not shown) is inhaling. Exhaled breath sample travels along the receiving section 106 to a first downward section 113 which is followed by a trapping section 111. Between the first downward section 113 and the trapping section 111 is a vent 108. When the subject is exhaling, the exhaled air forces the vent 108 to open to allow the breath sample to travel through the first downward section 113, the vent 108 in the open position and into the trapping section 111. The breath sample leaves the trapping section 111 with only the gaseous phase to enter into the upward section 112 leaving behind any liquid in the trapping section 111. The gaseous phase of the breath sample continues in the tubular structure 101 to enter a second downward section 114 to arrive at a condensation chamber 119. In the condensation chamber 119 is a microcondenser 120 to cool down the gaseous phase of the breath sample to form droplets 126. Excess gaseous phase of the exhaled breath sample can escape the system 100 through an exhaled air outlet 116. A cooling system 122 helps to provide cooling effect for the microcondenser 120. A preferred cooling system 122 is a Peltier cooler, which requires connection to a power source (not shown). The droplets 126 are then collected in a condensation droplet collecting tubing 124 and the droplets 126 move along the droplet collecting tubing by means of an active push. The active push can be provided by a device such as a peristaltic pump (not shown). The droplets 126 are released from the droplet collecting tubing 124 to the cells 132 within the optically clear bottom plate 130. The cells 132 have a GPCR, a G-protein and a reporter. In the presence of a target molecule, the cells generate an emitted light 144, which can be luminescence or fluorescence depending on the reporter used. The plate is optically clear at the bottom to allow the emitted light 144 to be focused by an objective 146 to a photosensor 150, which converts the light signal to a digital signal to be detected by a computing device 160 to display the digital signal. In the embodiment wherein the emitted light is fluorescence, the system further comprises an excitation light source 140 providing excitation light 142 onto the cells. An appropriate filter (not shown) blocks out the excitation light to allow only the excitation light with the desirable excitation wavelength to pass through.

In a further embodiment, the present invention discloses a system for determining a disease state of a mammalian subject wherein the disease state exhibits a unique combination of water-soluble volatile organic marker metabolites present in a fluid sample from the subject, and the system comprising: (a) A receiver to receive the fluid sample into the system; (b) A separator separating the water-soluble volatile organic molecules from the fluid medium wherein the fluid medium is retained in the system or released from the system and the water-soluble volatile organic molecules are allowed to move forward within the system; (c) An optically clear bottom plate with a plurality of wells of live non-neuron cells each well having a single type of live non-neuron cell covered with a cell culture medium to keep the cell alive wherein (i) the water-soluble volatile organic molecules are allowed to disperse into the cell culture medium in the wells; (ii) the live non-neuron cell having a surface GPCR, a G-protein and a reporter wherein the GPCR is capable of binding to the target molecule to produce a light emission; and (iii) the non-neuron cells separately express a GPCR specific to each of the metabolites exhibited by the disease state; (b) A photosensor below the plate for detecting the light emission from each well; (c) A computing device connecting to the photosensor having a display for displaying the light emission from each well wherein the presence of a light emission indicates the presence of the target molecule in the fluid sample and the presence of the entire combination of the marker metabolites indicate the presence of the disease state of the subject.

In another preferred embodiment, the system provides a specific odorant fingerprint on the detection array. The system comprises an array of n×m wells containing the cells with a predominant olfactory receptor. Ideally, each well contains a single type of olfactory neuron with a predominant olfactory receptor.

An odorant or VOC binding to an odorant receptor on the cell surface will activate a signaling pathway within the cell, which can trigger fluorescence.

If one sets up an array of patches of cells, each containing a different cell or group of cells, with each cell in a well expressing a specific odorant receptor, then an odorant will bind differentially across the wells. Thus, each well will have a different response to a set of VOCs.

Through repeated delivery of a single VOC or set of VOC, we can get a series of relative signals across the array. The signals can be represented in a matrix where each element represents a real value qᵢⱼ where q is the action potential and i and j represent the position of the array on the wells.

$$\begin{matrix}q 00 & q 01 & q 02 & q 03 & … & q 0 n \\ q 10 & q 11 & q 12 & q 13 & … & q 1 n \\ q 20 & q 21 & q 22 & q 23 & … & q 2 n \\ q 30 & q 31 & q 32 & q 33 & … & q 3 n \\ … & & & & & \\ qm 0 & qm 1 & qm 2 & qm 3 & … & qmn\end{matrix}$$

A single compound will bind to different receptors at different rates since the binding to G protein coupled receptors (GPCRs) is a 3-dimensional binding event. Binding occurs in the binding site of the receptor (Keller et al., 2017).

It is the combination of features on the compound that provide a 3-dimensional ligand “shape” or conformation to bind inside the GPCR binding pocket. Thus, different parts of the ligand will bind to different receptors differently and trigger different signals in different cell populations on the array of wells.

For example, 3-heptanone will bind differentially to receptors OR2W1, MOR272-1, MOR271-1, OR1A1 and MOR203-1 (H. Saito et al., 2009). For example, 2-octanone will bind differentially to receptors OR2W1, MOR272-1, OR1A1, MOR203-1 (Saito et al., 2009).

A single compound at a given concentration will trigger a relatively fixed set of values in this matrix. This can be used as a fingerprint for that particular compound.

A set of compounds (related or unrelated) will have a particular fingerprint when mapped against a particular set of receptors in an array of cells. This fingerprint for a set of compounds represents an overlapping set of the individual compounds binding. That is, one would expect the individual compounds in the set of compounds to bind to more than one receptor in different ways. The entire set would be additive across the array; however, the signals from some would mask the signals from others. Each combination of compounds would have a unique signature across the entire array. The size of the matrix of the array can vary, which may be from 1×1 to about 20×20 to about 100×100, or even higher.

The present application is also related to a method for determining a disease state of a mammalian subject wherein the disease state exhibits a unique combination of water-soluble volatile organic marker metabolites present in a fluid sample, the steps comprising;
a. Providing a fluid sample from the subject;
b. Contacting the fluid sample to an array of wells within a plate with an optically clear bottom, each well containing live non-neuron cells covered with a cell culture medium to keep the cells live, wherein the live non-neuron cell having a surface GPCR, a G-protein and a reporter wherein the GPCR is capable of binding to the target molecule to produce a light emission signal in the presence of the G-protein and reporter;
c. Detecting the emitted light signal with a photosensor below the plate; and
d. Displaying the light signals by a computing device connected to the photosensor wherein a pattern of combination of activated GPCRs results in recognition of a specific disease state.

Table 1 lists a set of metabolites associated with hepatitis C-related liver cirrhosis and hepatocellular carcinoma (Nomair et al. 2019).

TABLE 1
Metabolites negatively associated with hepatitis C-related
liver cirrhosis and hepatocellular carcinoma.

Dihydroxy acetophenone
Trisiloxane
benzenedicarboxaldehyde
carbamic acid
3-ethyl 2-methylhexane
silane
pyridinecarbonitrile
caprylic acid
oxomalonic acid
oxalic acid
neohexane
enanthic acid
caproic acid
butane
lodododecane
valeric acid
glutaric acid
methoxy benzoic acid
ethanol
hypoxanthine
arachidic acid
palmitic acid
pentadecylic acid
heptadecanoic acid
proprionic acid
capric acid
oleic acid
stearic acid
glycine
methionine
1-leucine
butylhydroquinone
acrylic acid
isophthalic acid

Table 2 lists a set of metabolites associated with a viral infection state. Consider headspace volatiles associated with influenza infection of lymphocytes (Aksenov et al. 2014).

TABLE 2a
Tentative volatile compounds linked to mild H1N1 infection

2-methoxy-ethanol
propanoic acid, ethyl ester
butanoic acid, 2-methyl-, ethyl ester

TABLE 2b
Tentative VOCs differentiating mild vs. full H1N1 infection

2-methoxy-ethanol
thiirane
hexan-3-one, 5-methyl-
heptan-3-one
octan-2-one

TABLE 2c
Tentative VOCs linked to H1N1, H6N2 or H9N2 infection

2-methoxy-ethanol
propanoic acid, ethyl ester
butanoic acid, 2-methyl-, methyl ester
butanoic acid, 2-methyl-, ethyl ester
1-phenylbut-1-ene

TABLE 4d
Tentative VOCs differentiating different, viral strains

2-methoxy-ethanol
thiirane
hexan-3-one, 5-methyl-
heptan-3-one
octan-2-one

Table 3 is a set of metabolites associated with a human rhinovirus (HRV) infection state as detected in cultured primary human tracheobronchial cells. Scichivo et al. (2014) determined differential expression of compounds such as aliphatic alcohols, branched hydrocarbons, and dimethyl sulfide by the infected cells. VOCs previously associated with oxidative stress and bacterial infection.

TABLE 3a
small molecule VOCs seen only in infected
cells 12-hours post-infection

acetone
organic ester, likely aromatic
molecule not identified, but likely an aliphatic hydrocarbon
aliphatic hydrocarbon
aliphatic compound, e.g., E-7-tetradecenol

TABLE 3b
small molecule VOCs seen only in infected
cells 24-hours post-infection

molecule not identified, but likely an aliphatic hydrocarbon
hydrocarbon, e.g., 2,3,4-trimethyl-hexan

TABLE 3c
small molecule VOCs seen only in infected
cells 24-hours post-infection

dimethyl sulfide
acetic acid
molecule not identified, but likely an aliphatic hydrocarbon

Table 4 is a list of VOCs associated with lung cancer.

TABLE 4a
Lung Cancer Metabolites disclosed by Jia et al. (Metabolites
2019, 9, 52; doi: 10.3390/metabo9030052)

1-butanol
n-octane
isopropylamine
methylcyclopentane
1,2,4-trimethylbenzene
2-methylpentane
decane
propylbenzene
ethylbenzene
pentane
heptane
1,2,3-trimethylbenzene
acetophenone
2,2,4,6,6-pentamethylheptane
3-methylhexane
2-methylhexane
2-pentadecanone
nonadecane
pentanal
octanal
nonanal
4-heptanone
thiophene

TABLE 4b
Lung Cancer Metabolites disclosed by Ratiu et al. (J.
Clin. Med. 2021, 10, 32; doi: 10.3390/jcm 10010032)

limonene

TABLE 4c
Lung Cancer Metabolites disclosed by Dent et al. (J Thorac Dis 2013;
5(S5): S540-S550; doi: 10.3978/j.issn.2072-1439.2013.08.44)

2-decanone
2-undecanone
2-nonanone
1-hexanone
2-3-butadione

### EXAMPLES

### Example 1: Gas Phase Detection of Target Molecules

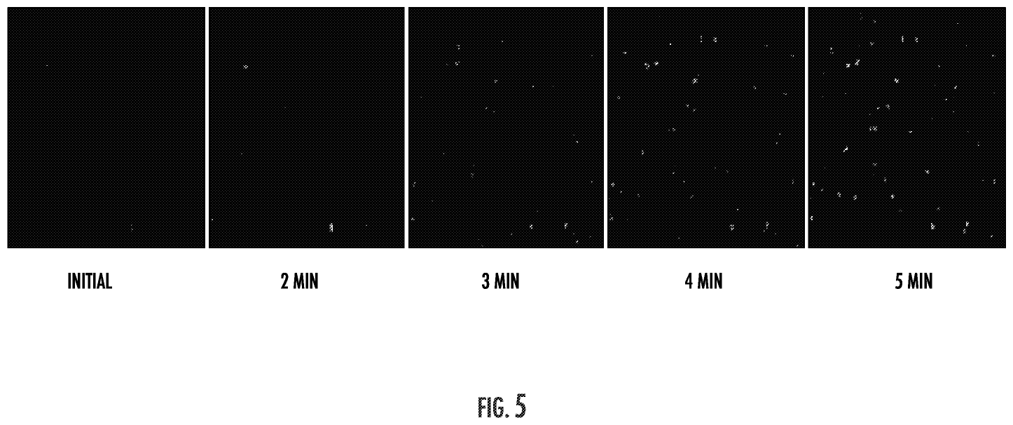

FIG. 5 shows the cells responding to lung cancer metabolite propylbenzene in a gas phase. Transfected human embryonic kidney cells were seeded at 60,000 cells per well in a poly-D-lysine coated 96-well plate. The exogenous ethylene benzene responsive GPCR and calcium reporter were present in the cytosol. Pure ethylene benzene was deposited as a hanging 5 μL droplet on the plate lid. Once the plate was placed over the well, there was no direct contact between the propylbenzene and the culture medium overlaid cells. Within minutes, the amount of volatile ethylene benzene evaporating from the pure 5 μL droplet and redissolving into the culture medium was sufficient to trigger the exogenous GPCR and induce a measurable fluorescence increase of the calcium reporter.

### Example 2: Luminescent Assay

Cells are seeded at a density of 15,000 cells per well in a solid white 96-well microplate and allowed to adhere prior to be transfected with different plasmids including but not limited to 10 ng GPCR-expressing plasmid and 10 ng cAMP-responsive luciferase reporter plasmid. The day following the transfection, the receptor-expressing cells are then stimulated with the ligands being tested. After a period to 5.5 hours following the stimulation, the cells are lysed and the luciferase reporter activity is measured following the manufacturer protocol. The amount of luciferase activity is proportional to the ligand-mediated activation level of the exogenous GPCR.

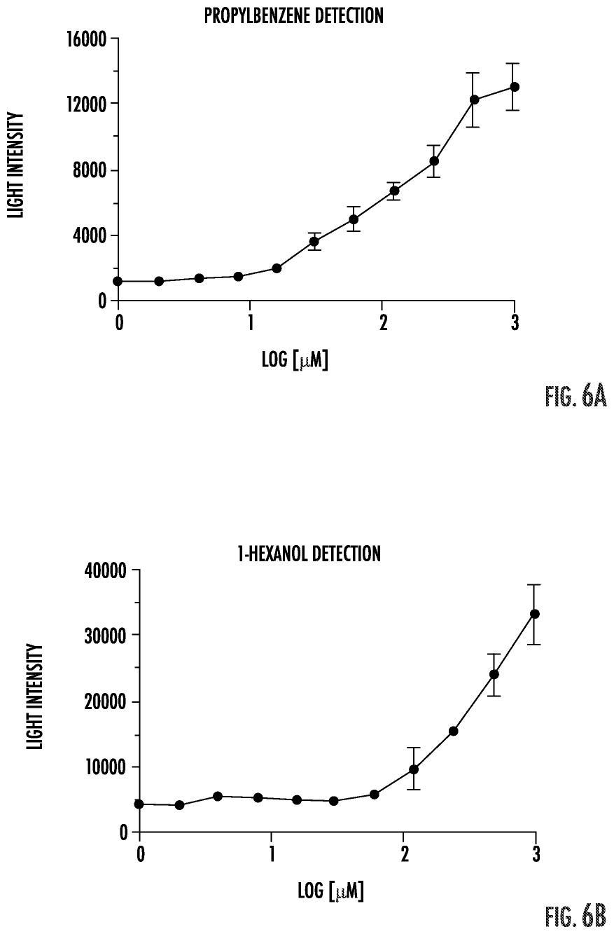

FIG. 6 is the result of luminescent assay showing dose response curves of OR1A1 for propylbenzene in FIG. 6A and 1-hexanol in FIG. 6B. The cells produce light by expressing a luciferase enzyme as a reporter readout from a cAMP secondary messenger. The ligand-mediated GPCR activation leads to intracellular cAMP accumulation which triggers a luciferase activity increase which is measured.

### Example 3: Fluorescence Assay

Human embryonic kidney cells are seeded at a density of 60,000 cells per well in a 96-well plate with an optically clear bottom to allow for fluorescent imaging. The cells are allowed to adhere prior to being transfected with different plasmids including but not limited to 10 ng GPCR-expressing plasmid and 60 ng of Ca²⁺-responsive fluorescence reporter plasmid. The day following the transfection, the receptor-expressing cells are then stimulated with the ligand being tested. The live cells are stimulated with excitation light and fluorescent light is collected to measure the Ca²⁺ reporter activity. The fluorescence of the cells increases from a baseline level to a higher level following the intracellular build up of secondary messengers such as Ca²⁺. The amount of fluorescence activity is proportional to the ligand-mediated activation level of the exogenous GPCR.

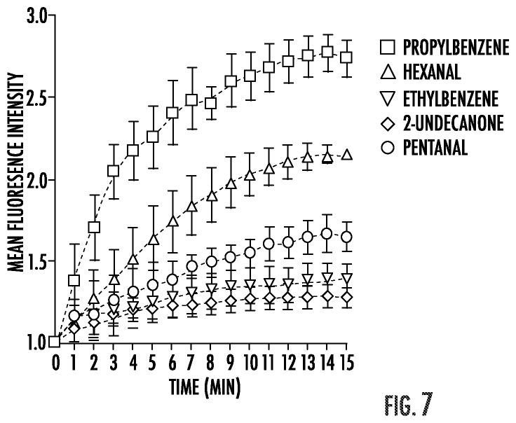

FIG. 7 shows the results of fluorescence assay. Time series of fluorescence following exposure to 1 mM of different lung cancer metabolites. Metabolite-induced human receptor OR1A1 activity leads to an increase in intracellular Ca²⁺ ions which triggers an increase of fluorescence of the cytosolic reporter.

Unless specifically defined otherwise, all technical and scientific terms used herein shall be taken to have the same meaning as commonly understood by one of ordinary skill in the art (e.g., in cell culture, molecular genetics, biosensors, G-coupled protein receptor biology, immunology, immunohistochemistry, protein chemistry, and biochemistry).

While the present invention is described in connection with what is presently considered to be the most practical and preferred embodiments, it should be appreciated that the invention is not limited to the disclosed embodiments and is intended to cover various modifications and equivalent arrangements included within the spirit and scope of the claims. Modifications and variation as defined in the claims. The appended claims should be construed broadly and in a manner consistent with the spirit and scope of the invention herein. It is understood that, given the above description of the embodiments of the invention, various modifications may be made by one skilled in the art. Such modifications are intended to be encompassed by the claims below.

The use of the terms “a” and “an” and “the” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually received herein. All methods described herein can be performed in any suitable order unless otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and biotechnology arts. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

As used in this application, the terms “computer” and “system” are intended to refer to a computer-related entity, either hardware, a combination of hardware and software, software, or software in execution. For example, a component can be, but is not limited to being, a process running on a processor, a processor, an object, an executable, a thread of execution, a program, and/or a computer. By way of illustration, both an application running on a server and the server can be a component. One or more components can reside within a process and/or thread of execution, and a component can be localized on one computer and/or distributed between two or more computers.

All publications and patent applications mentioned in this specification are herein incorporated by reference to the same extent as if each individual publication or patent application was specifically and individually indicated to be incorporated by reference.

### Additional Embodiments of Invention

The following disclosure includes additional and/or alternate features and embodiments and methods for the disclosed system.

For example, in an alternate embodiment of the claimed system wherein fluid sample is a liquid sample and the separator is a porous barrier which allows the water-soluble volatile organic molecules to pass through the porous membrane to reach the wells into the cell culture medium and subsequently interacting with the surface GPCR of the live non-neuron cells while the liquid medium is excluded from reaching the wells.

Additionally, wherein the fluid sample is a gaseous sample, the separator comprises:
a. tubing having a proximal end and a distal end and wherein the proximal end serves as the receiver or is connected to the receiver and the distal end is situated above the optically clear plate with a plurality of wells of cells;
b. a resin portion of the tubing situated at the distal end of the tubing filled with a resin within the tubing and the resin having the capability of trapping water-soluble volatile organic molecules;
c. a vent below the resin portion of the tubing and above the plate with the plurality of wells and the vent having an open position and a close position wherein when the vent is in the close position, the gaseous sample is kept within the tubing and passes through resin wherein the water-soluble volatile organic molecules are adsorbed by the resin; and
d. a heating coil surrounding the resin portion of the tubing wherein the water-soluble volatile organic molecules are released from the resin to disperse into the cell culture medium in the wells when the heating coil is turned on and when the vent is in the open position.

Specific embodiments of the claimed system may include wherein the vent is controlled by a button wherein the vent is closed when the button is depressed and the vent is open when the button is released.

Specific embodiments of the claimed system may also include wherein the fluid sample is a gaseous sample from an exhaled breath of a mammalian subject wherein the separator separating the gaseous phase from the liquid phase comprises:
a. a tubular structure having a proximal end and a distal end and the tubular structure provides a convoluted path for the passage of the breath sample, the tubular structure comprises:
   i. a receiving section at the proximal end of the tubular structure serving as a receiver or connected to the receiver of the system to receive the breath sample from the subject;
   ii. an inhaled air inlet within the receiving section to let air into the tubular structure to provide air for the individual when the individual is inhaling;
   iii. a first downward section allowing the breath sample to move down;
   iv. a trapping section to trap the liquid phase from the breath sample which has moved down from the first downward section;
   v. a vent between the first downward section and the trapping section, the vent being in a close position when the individual is inhaling and in an open position when the individual is exhaling to allow the exhaled breath sample to move along the first downward section and through the open vent into the trapping section;
   vi. an upward section allowing the gaseous phase of the breath sample to move upward leaving the liquid phase of the breath sample behind within the trapping section; and
   vii. a second downward section at the distal end of the tubular structure allowing the gaseous phase of the breath sample to move down;
b. a condensation chamber connecting to the second downward section of the tubular structure;
c. an exhaled outlet in the condensation chamber to allow excessive exhaled sample to escape from the tubular structure and provide a push to move the exhaled breath sample along the tubular structure;
d. a microcondenser in the condensation chamber to condense the gaseous phase of the breath sample into liquid droplets;
e. a cooling system below the microcondenser to lower the temperature of the microcondenser to facilitate the condensation of the gaseous phase of the breath sample on the microcondenser; and
f. a condensation droplet collecting tubing to collect the liquid droplets from the microcondenser, move the droplets along the collecting tubing and release the droplets from the tubing onto the cells in the plate wherein the moving of the droplets along the collecting tubing and releasing the droplets from the collecting tubing is by means of an active push.

Specific embodiments of the claimed system may also include wherein the cooling system is ice, dry ice, a fan or a peltier cooler, or wherein the active push is a peristaltic pump.

A method for determining a disease state of a mammalian subject wherein the disease state exhibits a unique combination of water-soluble volatile organic marker metabolites present in a fluid sample, the steps comprising;
a. providing a fluid sample from the subject;
b. contacting the fluid sample to an array of optically clear bottom wells containing live non-neuron cells covered with a cell culture medium to keep the cells live, wherein each well contains a unique live non-neuron cell having a surface. GPCR, a G-protein and a reporter wherein the GPCR is activated when it binds to the target molecule to trigger a cascade of intracellular events to produce a light emission signal;
c. detecting the emitted light signal with a photosensor below the wells; and
d. displaying the light signals by a computing device connected to the photosensor wherein a pattern of combination of activated GPCRs results in recognition of a specific disease state.

Also disclosed is a system for determining a disease state of a mammalian subject wherein the disease state exhibits a unique combination of water-soluble volatile organic marker metabolites present in a fluid sample from the subject, the system comprising:
a. a receiver to receive the fluid sample into the system;
b. a separator separating the water-soluble volatile organic molecules from the fluid medium wherein the fluid medium is retained in the system or released from the system and the water-soluble volatile organic molecules are allowed to move forward within the system;
c. an optically clear bottom plate with a plurality of wells of live non-neuronal cells each well having a single type of live non-neuron cell covered with a cell culture medium to keep the cell alive wherein
   i. the water-soluble volatile organic molecules are allowed to disperse into the cell culture medium in the wells;
   ii. the live non-neuron cell having a surface GPCR, a G-protein and a reporter wherein the GPCR is activated when it binds to the target molecule to trigger a cascade of events to produce a light emission; and
   iii. the non-neuron cells separately express a GPCR specific to each of the metabolites exhibited by the disease state;
d. a photosensor below the plate for detecting the light emission from the cells in the wells; and
e. a computing device connecting to the photosensor having a display for displaying the light emission from each well wherein the presence of a light emission indicates the presence of the target molecule in the fluid sample and the presence of the entire combination of the marker metabolites indicate the presence of the disease state of the subject.

Specific embodiments of the claimed system may also include wherein the wells are arranged in arrays, and wherein the computing device displays a pattern of a combination of activated GPCRs resulting in recognition of a specific disease state.

### REFERENCES

1. Saito H, Chi Q, Zhuang H,‡, Matsunami H, and Mainland J D. Odor Coding by a Mammalian Receptor Repertoire. Sci Signal, 2009 Mar. 3: 2(60):ra9. doi: 10:1126/scisignal.20000016.
2. Binkowski B F, Butler B L, Stecha P F, Eggers C T, Otto P. Zimmerman K, Vidugiris G, Wood M G, Encell L P, Fan F, Wood K V. A luminescent biosensor with increased dynamic range for intracellular cAMP. ACS Chem Biol. 011 November 18; 6(11):1193-7.doi: 10.1021/cb200248h. Epub 2011 Sep. 22.
3. Dana H, Sun Y, Mohar B, Hulse B K, Kerlin A M, Hasseman J P, Tsegaye G, Tsang A, Wong A, Patel R, Macklin J J, Chen Y, Konnerth A, Jayaraman V, Looger L L, Schreiter E R, Svoboda K, Kim D S. High-performance calcium sensors for imaging activity in neuronal populations and microcompartments. Nature Methods: 16, 649-657(2019).
4. Geijsen A J M R, Brezina S, Keski-Rahkonen P, Baierl A, Bachleitner-Hofmann T, Bergmann M M, Boehm J, Brenner H, Chang-Claude J, van Duijnhoven F J B, Gigic B, Gumpenberger T, Hofer P, Hoffneister M, Holowatyj A N, Karner-Hanusch J, Kok D E, Leeb G, Ulvik A, Robinot N, Ose J, Stift A, Schrotz-King P, Ulrich A B, Ueland P M, Kampman E, Scalbert A, Habermann N. Gsur A, Ulrich C M. Plasma metabolites associated with colorectal cancer: A discovery-replication strategy. Int J Cancer. 2019 Sep. 1; 145(5):1221-1231. doi: 10.1002/ijc.32146. Epub 2019 Feb. 14.
5. Geijsen A J M R, van Roekel E H, van Duijnhoven F J B, Achaintre D, Bachleitner-Hofmann T, Baierl A, Bergmann M M, Boehm J, Bours M J L, Brenner H, Breukink S O, Brezina S. Chang-Claude J, Herpel E, de Wilt J H W, Gicquiau A, Gigic B, Gumpenberger T, Hansson B M E, Hoffmeister M, Holowatyj A N, Karner-Hanusch J, Keski-Rahkonen P, Keulen E T P, Koole J L, Leeb G, Ose J, Schirmacher P, Schneider M A, Schrotz-King P, Stift A, Ulvik A, Vogelaar F J, Wesselink E, van Zutphen M, Gsur A, Habermann N, Kampman E, Scalbert A, Ueland P M, Ulrich A B, Ulrich C M, Weijenberg M P, Kok D E. Plasma metabolites associated with colorectal cancer stage: Findings from an international consortium. Int J Cancer. 2019 Sep. 8. doi: 10.1002/ijc.32666.
6. Keller A, Gerkin R C, Guan Y, Dhurandhar A, Turu G, Szalai B, Mainland J D, thara Y, Yu C W, Wolfinger R, Vens C, Schietgat L, De Grave K, Norel R; DREAM Olfaction Prediction Consortium, Stolovitzky G, Cecchi G, Vosshall L B, Meyer P. Predicting human olfactory perception from chemical features of odor molecules. Science. 2017 Feb. 24; 355(6327):820-826. doi: 10.1126/science.aal2014
7. Nomair A M, Madkour M A, Shamseya M M, Elsheredy H G, Shokr A. Profiling of plasma metabolomics in patients with hepatitis C-related liver cirrhosis and hepatocellular carcinoma. Clin Exp Hepatol. 2019 November; 5(4):317-326. doi: 10.5114/ceh.2019.89478. Epub 2019 Nov. 8. PubMed PMID: 31893244; PubMed Central PMCID: PMC6935851
8. Alexander A. Aksenov, Christian E. Sandrock, Weixiang Zhao, Shankar Sankaran, Michael Schivo, Richart Harper, Carol J. Cardona, Zheng Xing, and Cristina E. Davis; Cellular Scent of Influenza Virus Infection. Chembiochem. 2014 May 5; 15(7): 1040-1048. doi:10.1002/cbic.201300695
9. Saito H¹, Kubota M, Roberts R W, Chi Q, Matsunami H. RTP family members induce functional expression of mammalian odorant receptors. Cell. 2004 Nov. 24; 119(5):679-91.
10. Harumi Saito, Qiuyi Chi, Hanyi Zhuang, Hiro Matsunami, and Joel D. Mainland; Odor Coding by a Mammalian Receptor Repertoire. Sci Signal.; 2(60): ra9. doi:10.1126/scisignal.2000016
11. Schivo M, Aksenov A A, Linderholm A L, McCartney M M. Simmons J, Harper R W, Davis C E. Volatile emanations from in vitro airway cells infected with human rhinovirus. J Breath Res. 2014 September; 8(3):037110. doi: 10.1088/1752-7155/8/3/037110. Epub 2014 Sep. 4. PubMed PMID: 25189196: PubMed Central PMCID: PMC4182727.
12. Traxler S, Barkowsky G, Sas R, Klemenz A C, Patenge N, Kreikemeyer B, Schubert J K, Miekisch W. Volatile scents of influenza A and S. pyogenes (co-)infected cells. Sci Rep. 2019 Dec. 11; 9(1):18894. doi: 10.1038/s41598-019-55334-0. PubMed PMID: 31827195; PubMed Central PMCID: PMC6906285.
13. Traxler S, Bischoff A C, SaB R, Trefz P, Gierschner P, Brock B, Schwaiger T, Karte C, Blohm U, Schröder C. Miekisch W, Schubert J K. VOC breath profile in spontaneously breathing awake swine during Influenza A infection. Sci Rep. 2018 Oct. 5; 8(1):14857. doi: 10.1038/s41598-018-33061-2. PubMed PMID: 30291257; PubMed Central PMCID: PMC6173698.
14. Jia Z, Patr A, Kutty V A, Venkatesan T. Critical Review of Volatile Organic Compound Analysis in Breath and In Vitro Cell Culture for Detection of Lung Cancer. Metabolites 2019, 9, 52; doi:10.3390/metabo9030052.
15. Ratiu E A, Ligor T, Bocos-Bintintan V, Mayhew C S and Buszewski B. Volatile Organic Compounds in Exhaled Breath as Fingerprints of Lung Cancer, Asthma and COPD. J. Clin. Med. 2021, 10, 32; doi:10.3390/jcm10010032.
16. Dent A G, Sutedja T, Sutedja T G, Zimmerman P V. Exhaled breath analysis for lung cancer. J Thorac Dis 2013; 5(S5):S540-S550; doi:10.3978/j.issn.2072 1439.2013.08.44

What is claimed is:

1. A system for detecting a target molecule in a fluid sample from a subject, the fluid sample comprising molecules dissolved or suspended in a fluid medium, wherein the target molecule is a water-soluble volatile organic molecule, the system comprising:
a. a receiver to receive the fluid sample into the system;
b. a separator separating the water-soluble volatile organic molecules from the fluid medium wherein the fluid medium is retained in the system or released from the system and the water-soluble volatile organic molecules are allowed to move forward within the system;
c. an optically clear bottom plate with a plurality of wells of live non-neuron cells covered with a cell culture medium to keep the cells alive wherein the water-soluble volatile organic molecules are allowed to disperse into the cell culture medium covering the cells and wherein each well contains a unique cell having a surface G-protein coupled receptor (GPCR) capable of binding to the target molecule, an intracellular G-protein and an intracellular reporter and wherein the binding of the target molecule to the GPCR triggers a cascade of events within the cell to produce a secondary messenger signal which is captured by the reporter as an emitted light;
d. an objective to focus and direct the emitted light from the cells to a photosensor; and
e. a computing device connecting to the photosensor to receive the light emitting signal for further interpretation wherein the absence of a light signal indicates the absence of the target molecule and the presence of a light signal indicates the presence of the target molecule.

3. The system of claim 1 wherein the fluid sample is a liquid sample or a gaseous sample or a combination of liquid and gas.

4. The system of claim 2 wherein the fluid sample is a liquid sample and the liquid sample is a bodily fluid from a mammalian subject.

5. The system of claim 3 wherein the mammalian subject is a human subject.

6. The system of claim 1 wherein the fluid sample is prepared from an organic source.

7. The system of claim 5 wherein the organic source is a plant.

8. The system of claim 1 wherein the fluid sample is a gaseous sample from an exhaled breath of a mammalian subject wherein the sample comprises a gaseous phase containing small volatile organic molecules and a liquid phase of bodily fluid.

9. The system of claim 7 wherein the bodily fluid is saliva or mucus.

10. The system of claim 7 wherein the mammalian subject is a human subject.

11. The system of claim 1 wherein the cells are cultured at ambient temperature and ambient air without incubating the culture at 37° C. and 5% CO₂ by using a culture medium buffered by high buffering capacity carbon dioxide-independent buffer to maintain a pH of from about 7 to about 8 in the medium throughout the culture.

12. The system of claim 1 wherein the wells in the plate are arranged in arrays.

13. The system of claim 1 wherein the GPCR is an olfactory neuron GPCR.

14. The system of claim 1 wherein the GPCR, the G-protein or the reporter is an endogenous receptor naturally occurring in the cell or is an exogenous receptor expressed by the cell through genetic engineering and wherein the GPCR, the G-protein or reporter can be natural or synthetic.

15. The system of claim 1 wherein the GPCR is exogenous.

16. The system of claim 1 wherein the emitted light is luminescence or fluorescence.

17. The system of claim 15 wherein the emitted light is fluorescence, the system further comprises an excitation light source to generate the fluorescence.

18. The system of claim 1 wherein the reporter detects an increase in intracellular 3′, 5′-cyclic adenosine monophosphate (cAMP) or an increase in intracellular calcium.

19. The system of claim 17 wherein the reporter for detecting the increase in intracellular cAMP is a luciferase which generates luminescence light with the increase in intracellular cAMP.

20. The system of claim 1 wherein the reporter is a calcium reporter which detects the increase in intracellular calcium level to generate a fluorescence light upon excitation by an excitation light source.

21. The system of claim 19 wherein the calcium reporter is a genetically-encoded calcium indicator GCaMP.
